(19) United States
(12) Patent Application Publication
(10) Pub. No.: US 2007/0178106 A1
(43) Pub. Date: Aug. 2, 2007

(54) Compositions and methods for enhancing nk cell activity

(75) Inventor: Francois Romagne, La Ciotat (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: INNATE PHARMA, S.A., MARSEILLE FRANCE (FR)

(21) Appl. No.: 11/587,890

(22) PCT Filed: Apr. 29, 2005
(86) PCT No.: PCT/IB05/01434

§ 371 Date: Oct. 27, 2006

Related U.S. Application Data
(60) Provisional application No. 60/567,058, filed on Apr. 30, 2004.

(51) Int. Cl. A61K 39/395; C07H 21/04; C12P 21/06; C12N 5/06; C07K 16/30

(52) U.S. Cl. 424/155.1; 435/69.1; 435/338; 435/320.1; 530/388.8; 536/23.53

(57) ABSTRACT

The present invention relates to methods of treating malignancies and infections, methods of identifying patients suitable for treatment or for inclusion in clinical trials, and methods of producing antibodies for use in therapeutic applications. Generally, the present methods involve the use of antibodies that target activating receptors present on the surface of NK cells, and which at the same time interact with molecules present on the surface of malignant or infected target cells, thereby enhancing the therapeutic efficacy of the antibodies.

### FIELD OF THE INVENTION

The present invention relates to methods of treating malignancies and infections, methods of identifying patients suitable for treatments or for inclusion in clinical trials, and methods of producing antibodies for use in therapeutic applications. Generally, the present methods involve the use of antibodies that target activating receptors present on the surface of NK cells, and which at the same time help to guide activated NK cells to malignant or infected target cells, thereby enhancing their efficacy.

### BACKGROUND

Natural killer (NK) cells are a subpopulation of lymphocytes that are involved in non-conventional immunity. NK cells provide an efficient immunosurveillance mechanism by which undesired cells such as tumor or virally-infected cells can be eliminated. Characteristics and biological properties of NK cells include the expression of surface antigens including CD16, CD56 and/or CD57, the absence of the alpha/beta or gamma/delta TCR complex on the cell surface; the ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

NK cell activity is regulated by a complex mechanism that involves both activating and inhibitory signals. Several distinct NK-specific receptors have been identified that play an important role in the NK cell mediated recognition and killing of HLA Class I deficient target cells. These receptors, termed NKp30, NKp46 and NKp44, are members of the Ig superfamily. Their cross-linking, induced by specific mAbs, leads to a strong NK cell activation resulting in increased intracellular Ca⁺⁺ levels, triggering of cytotoxicity, and lymphokine release. Importantly, mAb-mediated activation of NKp30, NKp46, and/or NKp44 results in an activation of NK cytotoxicity against many types of target cells. These findings provide evidence for a central role of these receptors in natural cytotoxicity.

NK cells are negatively regulated by major histocompatibility complex (MHC) class I-specific inhibitory receptors (Kärre et al. (1986) Nature 319:675-8; Ohlen et al, (1989) Science 246:666-8). These specific receptors bind to polymorphic determinants of major histocompatibility complex (MHC) class I molecules or HLA and inhibit natural killer (NK) cell lysis. In humans, certain members of a family of receptors termed killer Ig-like receptors (KIRs) recognize groups of HLA class I alleles.

Fc receptors, such as Fc gamma receptors, are expressed on the surface of leukocytes. These receptors bind to the Fc portion of immunoglobulin (Ig), e.g. Fc gamma receptors bind to the Fc portion of IgG. This binding helps contribute to immune function by linking the recognition of antigens by antibodies with cell-based effector mechanisms. Different immunoglobulin classes trigger different effector mechanisms through the differential interaction of immunoglobulin Fc regions with specific Fc receptors (FcRs) on immune cells. Activating Fc gamma receptors include Fc gamma RI, Fc gamma RIIA, Fcgamma RIIC, and Fcgamma RIII A. Fc gamma RIIB is considered an inhibitory Fc gamma receptors. See, e.g., Woof et al. (2004) Nat Rev Immunol. 4(2):89-99; Baumann et al. (2003) Arch Immunol Ther Exp (Warsz) 51(6):399-406; and Pan et al. (2003) Chin Med J (Engl) 116(4):487-94).

### SUMMARY OF THE INVENTION

The present invention provides methods and compositions for producing antibodies useful for the treatment of disorders such as hematological malignancies and viral infections. The antibodies produced using the present methods are capable of activating NK cell cytotoxicity as well as promoting the interaction of NK cells with their target cells, thereby enhancing the ability of the cells to kill the targets. Methods of using the antibodies for the treatment of any of a number of disorders, including malignancies such as hematological malignancies, and infections such as viral infections, are also provided.

Accordingly, the present invention provides a method for producing an antibody suitable for use in treating malignancies or infections, the method comprising a) providing a human-suitable antibody that specifically binds to a human activating NK cell receptor, and b) assessing the ability of the antibody to specifically bind to a human Fc receptor, wherein a determination that the antibody can specifically bind to the receptor indicates that the antibody is suitable for use in treating the malignancies or infections.

In one embodiment, the human Fc receptor is an Fc gamma receptor, and the antibody is an IgG. In another embodiment, the Fc gamma receptor is selected from the group consisting of FCGR1A, FCGR1B, FCGR2A, FCGR2B, and FCGR3. In another embodiment, the Fc receptor is present on the surface of a cell or present within a cell membrane. In another embodiment, the malignancy is selected from the group consisting of follicular lymphoma, B cell lymphoma, macrophage tumor, acute monocytic leukemia, chronic myelomonocytic leukemia, chronic lymphocytic leukemia, mast cell malignancy, myeloid metaplasia, non-Hodgkin's lymphoma, and chronic myelocytic leukemia. In another embodiment, the malignancy or infection involves a cell selected from the group consisting of B cells, macrophages, mast cells, myeloid cells, monocytes, neutrophils, basophils, eosinophils, langerrhans cells, platelets and endothelial cells of the placenta. In another embodiment, the human-suitable antibody is derived from a mouse monoclonal antibody. In another embodiment, the activating NK cell receptor is NKp30 (Accession no. NP_{—}667341), NKp44 (Accession no. CAB39168), or NKp46 (Accession no. NP_{—}004820). The disclosures of the Genbank files corresponding to the aforementioned accession numbers are incorporated herein by reference. In another embodiment, the activating NK cell receptor is NKp30, and the human-suitable antibody is derived from a mouse monoclonal antibody selected from the group consisting of AZ20, A76, and Z25. In another embodiment, the activating NK cell receptor is NKp44, and the human-suitable antibody is derived from mouse monoclonal antibody Z231. In another embodiment, the activating NK cell receptor is NKp46, and the human-suitable antibody is derived from mouse monoclonal antibody BAB281.

In another aspect, the present invention provides a pharmaceutical composition comprising an antibody produced by any of the herein-described methods, and a pharmaceutically acceptable carrier.

In another aspect, the present invention provides a method for treating a patient with a malignancy selected from the group consisting of follicular lymphoma, B cell lymphoma, macrophage tumor, acute monocytic leukemia, chronic myelomonocytic leukemia, chronic lymphocytic leukemia, mast cell malignancy, myeloid metaplasia, non-Hodgkin's lymphoma, and chronic myelocytic leukemia, the method comprising administering to the patient a pharmaceutical composition comprising a human-suitable antibody that specifically binds to a human activating NK cell receptor, and a pharmaceutically acceptable carrier.

In another, related, aspect, the present invention provides a method for treating a patient with an infection or malignancy of cells selected from the group consisting of B cells, macrophages, mast cells, myeloid cells, monocytes, neutrophils, basophils, eosinophils, langerrhans cells, platelets and endothelial cells of the placenta, the method comprising administering to the patient a pharmaceutical composition comprising a human-suitable antibody that specifically binds to a human activating NK cell receptor, and a pharmaceutically acceptable carrier.

In one embodiment of either of these aspects, the human activating NK cell receptor is selected from the group consisting of NKp30, NKp44, and NKp46. In another embodiment, the activating NK cell receptor is NKp30, and the human-suitable antibody is derived from a mouse monoclonal antibody selected from the group consisting of AZ20, A76, and Z25. In another embodiment, the receptor is NKp44, and the human-suitable antibody is derived from the monoclonal antibody Z231. In another embodiment, the receptor is NKp46, and the human-suitable antibody is derived from the monoclonal antibody BAB281. In another embodiment, the antibody is an IgG. In another embodiment, the malignancy comprises cells expressing an Fc gamma receptor. In another embodiment, the Fc gamma receptor is selected from the group consisting of FCGR1A, FCGR1B, FCGR2A, FCGR2B, and FCGR3.

In another embodiment, the method further comprises a step in which the ability of the human-suitable antibody to interact with malignant cells taken from the patient is assessed prior to the administration of the composition, wherein a determination that the antibody specifically binds to the malignant cells indicates that the antibody is suitable for use in the treatment.

In another embodiment, the method further comprises a step in which the presence or absence of Fc receptors on the surface of malignant cells taken from the patient is assessed, wherein a detection of an Fc receptor on the surface of the malignant cells indicates that said antibody is suitable for use in the treatment. In one embodiment, the Fc receptor is an Fc gamma receptor. In another embodiment, the Fc gamma receptor is selected from the group consisting of FCGR1A, FCGR1B, FCGR2A, FCGR2B, and FCGR3.

In another embodiment, the human-suitable antibody contains a human Fc region. In another embodiment, the human-suitable antibody is a humanized or chimeric antibody.

A particular object of this invention thus an antibody, wherein the antibody is a humanized or otherwise human-suitable antibody that specifically binds to one or more human activating receptors on NK cells, and wherein the antibody is also specifically bound to by human Fc receptors, e.g. Fc gamma receptors, present on the surface of target cells. Kits comprising the present antibodies are also encompassed by the invention.

The invention also encompasses fragments and derivatives of the antibodies described having substantially the same antigen specificity and activity (e.g., which can cross-react with the parent antibody and which can activate NK cells as well as bind to human Fc receptors). Such fragments include, without limitation, Fab fragments, Fab′2 fragments, CDR and ScFv. Preferred are fragments that include, or have been derivatized to include, a human Fc domain or other motif so as to be recognizable by human Fc receptors.

The invention also relates to pharmaceutical compositions comprising an antibody as disclosed above or a fragment or derivative thereof, and a pharmaceutically acceptable carrier or excipient.

The invention also provides methods of regulating human NK cell activity in vitro, ex vivo or in vivo, comprising contacting human NK cells with an antibody or a fragment or derivative as defined above. Most preferred methods are directed at increasing the cytotoxic activity of human NK cells, most preferably ex vivo or in vivo, in a subject having a cancer, e.g. a hematological malignancy, or an infection.

### DETAILED DESCRIPTION OF THE INVENTION

Introduction

The present invention provides novel methods for producing antibodies suitable for the treatment of disorders such as malignancies, e.g., hematological malignancies, and infections, e.g. viral infections, in patients. Antibodies, antibody derivatives, or antibody fragments produced using the herein described methods are also encompassed, as are methods of treating patients using the antibodies. The antibodies described herein are capable of activating human NK cells in vivo, in particular by targeting one or more activating receptors on the surface of NK cells, such as NKp30, NKp44, and NKp46.

In addition to their ability to activate NK cells, the present antibodies also have an enhanced ability to promote the interaction between activated NK cells and their targets, thereby increasing the therapeutic efficacy of the antibodies for treating certain disorders. Notably, the present antibodies are especially useful for the treatment of certain hematological malignancies, such as follicular lymphoma, B cell lymphoma, macrophage tumors, acute monocytic leukemia, chronic myelomonocytic leukemia, chronic lymphocytic leukemia, mast cell malignancy, myeloid metaplasia, and chronic myelocytic leukemia, or any infection or malignancy involving any of the following cell types: B cells, macrophages, mast cells, myeloid cells, monocytes, neutrophils, basophils, eosinophils, langerrhans cells, platelets and endothelial cells of the placenta. In one embodiment, the activated NK cells have an enhanced ability to target cells that express an Fc receptor, preferably an Fc gamma receptor.

Without being bound by the following theory, it is believed that the particular effectiveness of the present antibodies is due at least in part to the ability of Fc receptors on the surface of target cells, e.g. Fc gamma receptors, to interact with the NK cell activating antibodies themselves. In this way, it is believed that the activated NK cells are brought into close proximity with their target cells via the two reactive portions of the antibody (e.g. the antigen-recognizing domain and the Fc domain), thereby enhancing the efficiency of the treatment.

Generally, the present methods involve providing monoclonal antibodies specific for one or more activating receptors on the surface of NK cells, such as NKp30, NKp44, or NKp46, and which are capable of activating the NK cells. The antibodies are then rendered suitable for use in humans, e.g., by producing humanized, chimeric, or human antibodies. These human-suitable antibodies are then assessed for their ability to interact with target cells, preferably target cells that express one or more human Fc receptors, such as Fc gamma receptors. Human-suitable antibodies that interact specifically with target cells are then selected for use in methods for the treatment of certain disorders, such as hematological malignancies or viral infections, preferably malignancies or infections involving Fc gamma receptor expressing cells.

Definitions

As used herein, the following terms have the meanings ascribed to them unless specified otherwise.

As used herein, “NK” cells refers to a sub-population of lymphocytes that is involved in non-conventional immunity. NK cells can be identified by virtue of certain characteristics and biological properties, such as the expression of specific surface antigens including CD16, CD56 and/or CD57, the absence of the alpha/beta or gamma/delta TCR complex on the cell surface, the ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG and/or IgM are the preferred classes of antibodies employed in this invention, with IgG being particularly preferred, because they are the most common antibodies in the physiological situation, because they are most easily made in a laboratory setting, and because IgGs are specifically recognized by Fc gamma receptors. Preferably the antibody of this invention is a monoclonal antibody. Particularly preferred are humanized, chimeric, human, or otherwise-human-suitable antibodies.

The term “specifically binds to” means that an antibody can bind preferably in a competitive binding assay to the binding partner, e.g. an activating NK receptor such as NKp30, NKp44, or NKp46, or a human Fc gamma receptor, as assessed using either recombinant forms of the proteins, epitopes therein, or native proteins present on the surface of isolated NK or relevant target cells. Competitive binding assays and other methods for determining specific binding are further described below and are well known in the art.

A “human-suitable” antibody refers to any antibody, derivatized antibody, or antibody fragment that can be safely used in humans for, e.g. the therapeutic methods described herein. Human-suitable antibodies include all types of humanized, chimeric, or fully human antibodies, or any antibodies in which at least a portion of the antibodies is derived from humans or otherwise modified so as to avoid the immune response that is provoked when native non-human antibodies are used.

By “immunogenic fragment”, it is herein meant any polypeptidic or peptidic fragment which is capable of eliciting an immune response such as (i) the generation of antibodies binding said fragment and/or binding any form of the molecule comprising said fragment, including the membrane-bound receptor and mutants derived therefrom, (ii) the stimulation of a T-cell response involving T-cells reacting to the bi-molecular complex comprising any MHC molecule and a peptide derived from said fragment, (iii) the binding of transfected vehicles such as bacteriophages or bacteria expressing genes encoding mammalian immunoglobulins. Alternatively, an immunogenic fragment also refers to any construction capable to elicit an immune response as defined above, such as a peptidic fragment conjugated to a carrier protein by covalent coupling, a chimeric recombinant polypeptide construct comprising said peptidic fragment in its amino acid sequence, and specifically includes cells transfected with a cDNA of which sequence comprises a portion encoding said fragment.

For the purposes of the present invention, a “humanized” antibody refers to an antibody in which the constant and variable framework region of one or more human immunoglobulins is fused with the binding region, e.g. the CDR, of an animal immunoglobulin. Such humanized antibodies are designed to maintain the binding specificity of the non-human antibody from which the binding regions are derived, but to avoid an immune reaction against the non-human antibody.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. In preferred embodiments of the present invention, the chimeric antibody nevertheless maintains the Fc region of the immunoglobulin, preferably a human Fc region, thereby allowing interactions with human Fc receptors on the surface of target cells. Within the context of this invention, “active” or “activated” NK cells designate biologically active NK cells, more particularly NK cells having the capacity of lysing target cells. For instance, an “active” NK cell is able to kill cells that express an NK activating receptor-ligand and fails to express “self” MHC/HLA antigens (KIR-incompatible cells). Examples of suitable target cells for use in redirected killing assays are P815 and K562 cells, but any of a number of cell types can be used and are well known in the art (see, e.g., Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8:1131-1135). “Active” or “activated” cells can also be identified by any other property or activity known in the art as associated with NK activity, such as cytokine (e.g. IFN-γ and TNF-α) production of increases in free intracellular calcium levels.

As used herein, the term “activating NK receptor” refers to any molecule on the surface of NK cells that, when stimulated, causes a measurable increase in any property or activity known in the art as associated with NK activity, such as cytokine (e.g. IFN-γ and TNF-α) production, increases in intracellular free calcium levels, or the ability to target cells in a redirected killing assay as described, e.g. elsewhere in the present specification. Examples of such receptors include NKp30, NKp44, and NKp46. Methods of determining whether an NK cell is active or not are described in more detail below.

The terms “isolated” “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The term “biological sample” as used herein includes but is not limited to a biological fluid (for example serum, lymph, blood), cell sample or tissue sample (for example bone marrow).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Producing Monoclonal Antibodies Specific for Activating NK Cell Receptors

The antibodies of this invention may be produced by any of a variety of techniques known in the art. Typically, they are produced by immunization of a non-human animal, preferably a mouse, with an immunogen comprising an activating receptor present on the surface of NK cells. The activating receptor may comprise entire NK cells or cell membranes, the full length sequence of a receptor such as NKp30 (see, e.g., PCT WO 01/36630, the disclosure of which is herein incorporated by reference in its entirety), NKp44 (see, e.g., Vitale et al. (1998) J. Exp. Med. 187:2065-2072, the disclosure of which is herein incorporated by reference in its entirety), or NKp46 (see, e.g., Sivori et al. (1997) J. Exp. Med. 186:1129-1136; Pessino et al. (1998) J. Exp. Med. 188:953-960; the disclosures of which are herein incorporated by reference in their entireties), or a fragment or derivative thereof, typically an immunogenic fragment, i.e., a portion of the polypeptide comprising an epitope exposed on the surface of the cell expressing any of these receptors, or any other receptor whose stimulation leads to the activation of NK cells. Such fragments typically contain at least 7 consecutive amino acids of the mature polypeptide sequence, even more preferably at least 10 consecutive amino acids thereof. They are essentially derived from the extracellular domain of the receptor. It will be appreciated that any receptor present on the surface of NK cells that, upon stimulation, leads to the activation of the cells as measured by cytotoxicity, increase in intracellular free calcium levels, cytokine production, or any other method known in the art can be used for the generation of antibodies. In preferred embodiments, the activating NK cell receptor used to generate antibodies is a human receptor.

In one embodiment, the immunogen comprises a wild-type human NKp30, NKp44, or NKp46 polypeptide in a lipid membrane, typically at the surface of a cell. In a specific embodiment, the immunogen comprises intact NK cells, particularly intact human NK cells, optionally treated or lysed. Examples of preferred isolated antibodies of the invention include isolated antibodies that are directed against at least one isolated amino acid compound of the invention, and that can induce an increase of at least about 4, preferably at least about 5, more preferably at least about 6 times, of the natural cytotoxicity triggered by a NK cell placed in the presence of a target cell in a 1:1 ratio.

Most preferred isolated antibodies of the invention are directed against at least one isolated amino acid compound of the invention, do not bind to any T or B cell surface molecule, and can induce an increase of at least about 4, preferably at least about 5, more preferably at least about 6 times, of the natural cytotoxicity triggered by a NK cell placed in the presence of a target cell in a 1:1 ratio

Antibodies can exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). In preferred embodiments, fragments will naturally include, or will be modified to include, a human Fc region or other domain that allows specific recognition of the antibody by human Fc receptors, e.g. Fe gamma receptors.

The preparation of monoclonal or polyclonal antibodies is well known in the art, and any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to desired polypeptides, e.g., activating NK cell receptors such as NKp30, NKp44, and NKp46. Also, transgenic mice, or other organisms such as other mammals, may be used to express humanized, chimeric, or similarly-modified antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). In one embodiment, the method comprises selecting, from a library or repertoire, a monoclonal antibody or a fragment or derivative thereof that cross reacts with at least one activating NK receptor such as NKp30, NKp44, or NKp46. For example, the repertoire may be any (recombinant) repertoire of antibodies or fragments thereof, optionally displayed by any suitable structure (e.g., phage, bacteria, synthetic complex, etc.).

The step of immunizing a non-human mammal with an antigen may be carried out in any manner well known in the art for (see, for example, E. Harlow and D. Lane, Antibodies: A Laboratory Manual, Cold Spring Harbor Laboratory Press, Cold Spring Harbor, N.Y. (1988)). Generally, the immunogen is suspended or dissolved in a buffer, optionally with an adjuvant, such as complete Freund's adjuvant. Methods for determining the amount of immunogen, types of buffers and amounts of adjuvant are well known to those of skill in the art and are not limiting in any way on the present invention.

Similarly, the location and frequency of immunization sufficient to stimulate the production of antibodies is also well known in the art. In a typical immunization protocol, the non-human animals are injected intraperitoneally with antigen on day 1 and again about a week later. This is followed by recall injections of the antigen around day 20, optionally with adjuvant such as incomplete Freund's adjuvant. The recall injections are performed intravenously and may be repeated for several consecutive days. This is followed by a booster injection at day 40, either intravenously or intraperitoneally, typically without adjuvant. This protocol results in the production of antigen-specific antibody-producing B cells after about 40 days. Other protocols may also be utilized as long as they result in the production of B cells expressing an antibody directed to the antigen used in immunization.

In an alternate embodiment, lymphocytes from an unimmunized non-human mammal are isolated, grown in vitro, and then exposed to the immunogen in cell culture. The lymphocytes are then harvested and the fusion step described below is carried out.

For monoclonal antibodies, which are preferred for the purposes of the present invention, the next step is the isolation of cells, e.g., lymphocytes, splenocytes, or B cells, from the immunized non-human mammal and the subsequent fusion of those splenocytes, or B cells, or lymphocytes, with an immortalized cell in order to form an antibody-producing hybridoma. Accordingly, the term “preparing antibodies from an immunized animal,” as used herein, includes obtaining B-cells/splenocytes/lymphocytes from an immunized animal and using those cells to produce a hybridoma that expresses antibodies, as well as obtaining antibodies directly from the serum of an immunized animal. The isolation of splenocytes, e.g., from a non-human mammal is well-known in the art and, e.g., involves removing the spleen from an anesthetized non-human mammal, cutting it into small pieces and squeezing the splenocytes from the splenic capsule and through a nylon mesh of a cell strainer into an appropriate buffer so as to produce a single cell suspension. The cells are washed, centrifuged and resuspended in a buffer that lyses any red blood cells. The solution is again centrifuged and remaining lymphocytes in the pellet are finally resuspended in fresh buffer.

Once isolated and present in single cell suspension, the antibody-producing cells are fused to an immortal cell line. This is typically a mouse myeloma cell line, although many other immortal cell lines useful for creating hybridomas are known in the art. Preferred murine myeloma lines include, but are not limited to, those derived from MOPC-21 and MPC-11 mouse tumors available from the Salk Institute Cell Distribution Center, San Diego, Calif. U.S.A., X63 Ag8653 and SP-2 cells available from the American Type Culture Collection, Rockville, Md. U.S.A. The fusion is effected using polyethylene glycol or the like. The resulting hybridomas are then grown in selective media that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

The hybridomas can be grown on a feeder layer of macrophages. The macrophages are preferably from littermates of the non-human mammal used to isolate splenocytes and are typically primed with incomplete Freund's adjuvant or the like several days before plating the hybridomas. Fusion methods are described, e.g., in (Goding, “Monoclonal Antibodies: Principles and Practice,” pp. 59-103 (Academic Press, 1986)), the disclosure of which is herein incorporated by reference.

The cells are allowed to grow in the selection media for sufficient time for colony formation and antibody production. This is usually between 7 and 14 days. The hybridoma colonies are then assayed for the production of antibodies that specifically recognize the desired substrate, e.g. activating NK cell receptors such as NKp30, NKp44, and/or NKp46. The assay is typically a colorimetric ELISA-type assay, although any assay may be employed that can be adapted to the wells that the hybridomas are grown in. Other assays include immunoprecipitation and radioimmunoassay. The wells positive for the desired antibody production are examined to determine if one or more distinct colonies are present. If more than one colony is present, the cells may be re-cloned and grown to ensure that only a single cell has given rise to the colony producing the desired antibody. Positive wells with a single apparent colony are typically recloned and re-assayed to insure only one monoclonal antibody is being detected and produced.

Hybridomas that are confirmed to be producing a monoclonal antibody of this invention are then grown up in larger amounts in an appropriate medium, such as DMEM or RPMI-1640. Alternatively, the hybridoma cells can be grown in vivo as ascites tumors in an animal.

After sufficient growth to produce the desired monoclonal antibody, the growth media containing monoclonal antibody (or the ascites fluid) is separated away from the cells and the monoclonal antibody present therein is purified. Purification is typically achieved by gel electrophoresis, dialysis, chromatography using protein A or protein G-Sepharose, or an anti-mouse Ig linked to a solid support such as agarose or Sepharose beads (all described, for example, in the Antibody Purification Handbook, Amersham Biosciences, publication No. 18-1037-46, Edition AC, the disclosure of which is hereby incorporated by reference). The bound antibody is typically eluted from protein A/protein G columns by using low pH buffers (glycine or acetate buffers of pH 3.0 or less) with immediate neutralization of antibody-containing fractions. These fractions are pooled, dialyzed, and concentrated as needed.

In preferred embodiments, the DNA encoding an antibody that binds a determinant present on an activating NK receptor is isolated from the hybridoma, placed in an appropriate expression vector for transfection into an appropriate host. The host is then used for the recombinant production of the antibody, variants thereof, active fragments thereof, or humanized or chimeric antibodies comprising the antigen recognition portion of the antibody. Preferably, the DNA used in this embodiment encodes an antibody that recognizes a determinant present on one or more human activating NK receptors such as NKp30, NKp44, or NKp46, and cause an activation of NK cells expressing the relevant receptors. Also preferably, the derivative or fragment contains a human or human-like Fc portion, e.g., one that is specifically recognized by human Fc receptors, such as Fc gamma receptors.

DNA encoding the monoclonal antibodies of the invention can be readily isolated and sequenced using conventional procedures (e.g., by using oligonucleotide probes that are capable of binding specifically to genes encoding the heavy and light chains of murine antibodies). Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant expression in bacteria of DNA encoding the antibody is well known in the art (see, for example, Skerra et al. (1993) Curr. Op. Immunol. 5:256; and Pluckthun (1992) Immunol. Revs. 130:151.

Antibodies may also be produced by selection of combinatorial libraries of immunoglobulins, as disclosed for instance in Ward et al. (1989) Nature 341:544.

In a specific embodiment, the antibody binds essentially the same epitope as any of the following monoclonal antibodies: AZ20, A76, Z25, Z231, or BAB281. Such antibodies are referred to herein as “AZ20-like antibodies,” “A76-like antibodies,” etc. The term “binds to substantially the same epitope or determinant as “the monoclonal antibody x means that an antibody “can compete” with x, where x is AZ20, A76, etc. The identification of one or more antibodies that bind(s) to substantially the same epitope as the monoclonal antibody in question can be readily determined using any one of variety of immunological screening assays in which antibody competition can be assessed. All such assays are routine in the art (see, e.g., U.S. Pat. No. 5,660,827, issued Aug. 26, 1997, which is specifically incorporated herein by reference). It will be understood that actually determining the epitope to which the antibody binds is not in any way required to identify an antibody that binds to the same or substantially the same epitope as the monoclonal antibody in question. The AZ20-producing hybridoma is deposited under number I-2576 at the C.N.C.M. on Nov. 8, 2000 (C.N.C.M., Institut Pasteur, 25 rue du Dr. Roux, F-75724 Paris Cedex 15, France. Bab281 is described in Sivori et al (1997) J. Exp. Med. 186:1129-1136; Z231 is described in Vitale et al. (1998) J. Exp. Med. 187:2065-2072; Z25 (and AZ20) is described in Pende et al. (1999) J Exp Med. 190(10):1505-1516.

For example, where the test antibodies to be examined are obtained from different source animals, or are even of a different Ig isotype, a simple competition assay may be employed in which the control (e.g. BAB281) and test antibodies are admixed (or pre-adsorbed) and applied to a sample containing the epitope-containing protein, e.g. NKp46in the case of BAB281. Protocols based upon ELISAs, radioimmunoassays, Western blotting and the use of BIACORE (as described, e.g., in the examples section) are suitable for use in such simple competition studies and are well known in the art.

In certain embodiments, one would pre-mix the control antibodies (e.g BAB281) with varying amounts of the test antibodies (e.g., 1:10 or 1:100) for a period of time prior to applying to the antigen (e.g. NKp46 epitope) containing sample. In other embodiments, the control and varying amounts of test antibodies can simply be admixed during exposure to the antigen sample. As long as one can distinguish bound from free antibodies (e.g., by using separation or washing techniques to eliminate unbound antibodies) and the control antibody from the test antibodies (e.g., by using species- or isotype-specific secondary antibodies or by specifically labeling the control antibody with a detectable label) one will be able to determine if the test antibodies reduce the binding of the control antibody to the antigen, indicating that the test antibody recognizes substantially the same epitope as the control. The binding of the (labeled) control antibodies in the absence of a completely irrelevant antibody would be the control high value. The control low value would be obtained by incubating the labeled control antibodies (e.g. BAB281) with unlabeled antibodies of exactly the same type (e.g. BAB281), where competition would occur and reduce binding of the labeled antibodies. In a test assay, a significant reduction in labeled antibody reactivity in the presence of a test antibody is indicative of a test antibody that recognizes the same epitope, i.e., one that “cross-reacts” with the labeled control antibody. Any test antibody that reduces the binding of the labeled control to each the antigen by at least 50% or more preferably 70%, at any ratio of control:test antibody between about 1:10 and about 1:100 is considered to be an antibody that binds to substantially the same epitope or determinant as the control. Preferably, such test antibody will reduce the binding of the control to the antigen by at least 90%.

In one embodiment, competition can be assessed by a flow cytometry test. Cells bearing a given activating receptor are incubated first with a control antibody that is known to specifically bind to the receptor (e.g., NK cells expressing NKp46, and the BAB281 antibody), and then with the test antibody that has been labeled with, e.g., a fluorochrome or biotin. The test antibody is said to compete with the control if the binding obtained with preincubation with saturating amounts of control antibody is 80%, preferably, 50, 40 or less the binding (mean of fluorescence) obtained by the antibody without preincubation with the control. Alternatively, a test antibody is said to compete with the control if the binding obtained with a labeled control (by a fluorochrome or biotin) on cells preincubated with saturating amount of antibody to test is 80%, preferably 50%, 40%, or less the binding obtained without preincubation with the antibody.

In one preferred example, a simple competition assay may be employed in which a test antibody is pre-adsorbed and applied at saturating concentration to a surface onto which is immobilized the substrate for the antibody binding, e.g. the NKp46 protein, or epitope-containing portion thereof, which is known to be bound by BAB281. The surface is preferably a BIACORE chip. The control antibody (e.g. BAB281) is then brought into contact with the surface at a substrate-saturating concentration and the substrate surface binding of the control antibody is measured. This binding of the control antibody is compared with the binding of the control antibody to the substrate-containing surface in the absence of test antibody. In a test assay, a significant reduction in binding of the substrate-containing surface by the control antibody in the presence of a test antibody is indicative of a test antibody that recognizes the same epitope, i.e., one that “cross-reacts” with the control antibody. Any test antibody that reduces the binding of the control antibody to the antigen-containing substrate by at least 30% or more preferably 40% is considered to be an antibody that binds to substantially the same epitope or determinant as the control antibody. Preferably, such test antibody will reduce the binding of the control antibody to the substrate by at least 50%. It will be appreciated that the order of control and test antibodies can be reversed, that is the control antibody is first bound to the surface and the test antibody is brought into contact with the surface thereafter. Preferably, the antibody having higher affinity for the substrate antigens is bound to the substrate-containing surface first since it will be expected that the decrease in binding seen for the second antibody (assuming the antibodies are cross-reacting) will be of greater magnitude. Further examples of such assays are provided in the Examples and in Saunal and Regenmortel, (1995) J. Immunol. Methods 183: 33-41, the disclosure of which is incorporated herein by reference.

In one embodiment, antibodies capable of interacting with multiple activating receptors on the NK cell surface, e.g. any combination of two or more of NKp30, NKp44, and NKp46, or any combination involving one ore more of these receptors and an additional activating receptor, may be obtained, particularly if it is ensured that the antibodies do not show excessive cross-reactivity with other, unrelated proteins. Monoclonal antibodies that recognize an epitope from an activating NK cell receptor, e.g. an NKp30, NKp44, or NKp46 epitope, will react with an epitope that is present on a high percentage NK cells e.g., at least about 70-90%, preferably about 80%, of such cells, but will not significantly react with CD3⁺ T cells or CD20⁺ B cells. In preferred embodiments, the antibody will also be nonreactive with monocytes, granulocytes, platelets, and red blood cells

Identifyng Antibodies that Activate NK Cells

Once an antibody that specifically recognizes one ore more activating receptors on NK cells, preferably human NK cells, is identified, it is first tested for its ability to activate intact NK cells, preferably human NK cells. Such antibodies are thereafter deemed “activating” antibodies, in the sense that they stimulate, at least partially, the activating signaling pathway mediated by the receptors. Specifically, the term “activate” NK cell activity, as used herein means, inter alia, the ability to increase of at least 20%, preferably at least 30%, 40% or 50% or more using any relevant assays or test, such as binding, expression-based, or cellular assays, many of which are available and suitable for the purposes of the present invention, and are well known to those of the art. Preferred antibodies of the invention can induce a statistically significant (p<0.05) increase in NK cell activation as assessed, e.g., by (i) natural cytotoxicity towards MHC class I negative targets, tumor cells, virally-infected cells, or allogeneic cells, (ii) cytotoxicity towards antibody-coated target cells, (iii) increases in intracytoplasmic Ca2+ concentration, (iv) induction of tyrosine phosphorylation of intracytoplasmic adaptor/effector molecules such as ZAP70, Syk, LAT, SLP76, Shc, Grb2, phospholipase C-gamma enzymes, phosphatidyl-inositol 3-kinases, (v) phosphorylation of receptor-associated transducing chains KARAP/DAP12 or CD3zeta or FcR gamma, (vi) cytokine secretion such as interferon gamma, tumor necrosis factors, IL5, IL10, chemokines (such as MIP-1alpha), TGFbeta, (vii) up- or down-regulation of NK cell surface molecules, such as CD69 and PEN5 respectively.

NK cell activation can be detected in any of a number of ways, e.g., by virtue of an increase in intracellular free calcium as described, e.g., in Sivori et al. (1997) J. Exp. Med. 186:1129-1136.

NK cell activity can also be assessed using a cell based cytotoxicity assays, e.g. measuring chromium release, such as assessing the ability of the antibody to stimulate NK cells to kill target cells such as P815, K562 cells, or appropriate tumor cells as disclosed in Sivori et al. (1997) J. Exp. Med. 186: 1129-1136; Vitale et al. (1998) J. Exp. Med. 187: 2065-2072; Pessino et al. (1998) J. Exp. Med. 188: 953-960; Neri et al. (2001) Clin. Diag. Lab. Immun. 8:1131-1135); Pende et al. (1999) J. Exp. Med. 190: 1505-1516), the entire disclosures of each of which are herein incorporated by reference. In a preferred embodiment, the antibodies cause at least a 10% augmentation in NK cytotoxicity, preferably at least a 40% or 50% augmentation in NK cytotoxicity, or more preferably at least a 70% augmentation in NK cytotoxicity.

NK cell activity can also be addressed using a cytokine-release assay, wherein NK cells are incubated with the antibody to stimulate the NK cells' cytokine production (for example IFN-γ and TNF-α production). In an exemplary protocol, IFN-γ production from PBMC is assessed by cell surface and intracytoplasmic staining and analysis by flow cytometry after 4 days in culture. Briefly, Brefeldin A (Sigma Aldrich) is added at a final concentration of 5 μg/ml for the last 4 hours of culture. The cells are then incubated with anti-CD3 and anti-CD56 mAb prior to permeabilization (IntraPrep™; Beckman Coulter) and staining with PE-anti-IFN-γ or PE-IgG1 (Phamhingen). GM-CSF and IFN-γ production from polyclonal activated NK cells are measured in supernatants using ELISA (GM-CSF: DuoSet Elisa, R&D Systems, Minneapolis, Minn.; IFN-γ: OptE1A set, Pharmingen).

In a preferred embodiment, the ability of the antibody to activate human NK cells is assessed, where an ability to activate human NK cells at least as well as non-human NK cells indicates that the antibodies are suitable for use in the present invention.

Producing Antibodies Suitable for Use in Humans

Once monoclonal antibodies are produced, generally in non-human animals, that can specifically bind to and stimulate human activating NK cell receptors, the antibodies are modified so as to make them suitable for therapeutic use in humans. For example, they may be humanized, chimerized, or selected from a library of human antibodies using methods well known in the art.

In one, preferred, embodiment, the DNA of a hybridoma producing an antibody of this invention, e.g. a BAB281-like antibody, can be modified prior to insertion into an expression vector, for example, by substituting the coding sequence for human heavy- and light-chain constant domains in place of the homologous non-human sequences (e.g., Morrison et al. (1984) Proc. Natl. Acad. Sci. 81:6851), or by covalently joining to the immunoglobulin coding sequence all or part of the coding sequence for a non-immunoglobulin polypeptide. In that manner, “chimeric” or “hybrid” antibodies are prepared that have the binding specificity of the original antibody. Typically, such non-immunoglobulin polypeptides are substituted for the constant domains of an antibody of the invention.

In one particularly preferred embodiment, the antibody of this invention is humanized. “Humanized” forms of antibodies according to this invention are specific chimeric immunoglobulins, immunoglobulin chains or fragments thereof (such as Fv, Fab, Fab′, F(ab′)₂, or other antigen-binding subsequences of antibodies) which contain minimal sequence derived from the murine or other non-human immunoglobulin. For the most part, humanized antibodies are human immunoglobulins (recipient antibody) in which residues from a complementary-determining region (CDR) of the recipient are replaced by residues from a CDR of the original antibody (donor antibody) while maintaining the desired specificity, affinity, and capacity of the original antibody. In some instances, Fv framework residues of the human immunoglobulin may be replaced by corresponding non-human residues. Furthermore, humanized antibodies can comprise residues that are not found in either the recipient antibody or in the imported CDR or framework sequences. These modifications are made to further refine and optimize antibody performance. In general, the humanized antibody will comprise substantially all of at least one, and typically two, variable domains, in which all or substantially all of the CDR regions correspond to those of the original antibody and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al., Nature, 321, pp. 522 (1986); Reichmann et al., Nature, 332, pp. 323 (1988); and Presta, Curr. Op. Struct. Biol., 2, pp. 593 (1992).

Methods for humanizing antibodies are well known in the art. Generally, a humanized antibody according to the present invention has one or more amino acid residues introduced into it from the original antibody. These murine or other non-human amino acid residues are often referred to as “import” residues, which are typically taken from an “import” variable domain. Humanization can be essentially performed following the method of Winter and co-workers (Jones et al. (1986) Nature 321:522; Riechmann et al. (1988) Nature 332:323; Verhoeyen et al. (1988) Science 239:1534 (1988)). In some cases, such “humanized” antibodies are chimeric antibodies (Cabilly et al., U.S. Pat. No. 4,816,567), wherein substantially less than an intact human variable domain has been substituted by the corresponding sequence from the original antibody. In practice, humanized antibodies according to this invention are typically human antibodies in which some CDR residues and possibly some FR residues are substituted by residues from analogous sites in the original antibody.

The choice of human variable domains, both light and heavy, to be used in making the humanized antibodies is very important to reduce antigenicity. According to the so-called “best-fit” method, the sequence of the variable domain of an antibody of this invention is screened against the entire library of known human variable-domain sequences. The human sequence which is closest to that of the mouse is then accepted as the human framework (FR) for the humanized antibody (Sims et al. (1993) J. Immunol., 151:2296; Chothia and Lesk (1987) J. Mol. Biol. 196:901). Another method uses a particular framework from the consensus sequence of all human antibodies of a particular subgroup of light or heavy chains. The same framework can be used for several different humanized antibodies (Carter et al. (1992) Proc. Natl. Acad. Sci. 89:4285; Presta et al. (1993) J. Immunol. 51:1993)).

It is further important that antibodies be humanized with retention of high affinity for one or more activating NK cell receptors, preferably human receptors, and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar. to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Another method of making “humanized” monoclonal antibodies is to use a XenoMouse® (Abgenix, Fremont, Calif.) as the mouse used for immunization. A XenoMouse is a murine host that has had its immunoglobulin genes replaced by functional human immunoglobulin genes. Thus, antibodies produced by this mouse or in hybridomas made from the B cells of this mouse, are already humanized. The XenoMouse is described in U.S. Pat. No. 6,162,963, which is herein incorporated in its entirety by reference. An analogous method can be achieved using a HuMAb-Mouse™ (Medarex).

Human antibodies may also be produced according to various other techniques, such as by using, for immunization, other transgenic animals that have been engineered to express a human antibody repertoire (Jakobovitz et al., Nature 362 (1993) 255), or by selection of ant body repertoires using phage display methods. Such techniques are known to the skilled person and can be implemented starting from monoclonal antibodies as disclosed in the present application.

The invention also encompasses fragments and derivatives of antibodies of this invention. “Immunoreactive fragments” comprise a portion of the intact antibody, generally the antigen binding site or variable region. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific antibodies formed from antibody fragments. For instance, Fab or F(ab′)2 fragments may be produced by protease digestion of the isolated antibodies, according to conventional techniques. In preferred embodiments, fragments or derivatives will comprise a human or human-like Fc portion, meaning a segment that is specifically recognized by human Fc receptors, such as Pc gamma receptors.

The antibodies of the present invention may also be derivatized to “chimeric” antibodies (immunoglobulins) in which a portion of the heavy and/or light chain is identical with or homologous to corresponding sequences in the original antibody, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., supra; Morrison et al. (1984) Proc. Natl. Acad. Sci. 81:6851).

Other derivatives within the scope of this invention include functionalized antibodies, i.e., antibodies that are conjugated or covalently bound to a toxin, such as ricin, diphtheria toxin, abrin and Pseudomonas exotoxin; to a detectable moiety, such as a fluorescent moiety, a radioisotope or an imaging agent; or to a solid support, such as agarose beads or the like. Methods for conjugation or covalent bonding of these other agents to antibodies are well known in the art.

Selecting Antibodies for Use in Treatment Methods

Once antibodies are obtained that are known to specifically bind to activating receptors on NK cells, preferably human NK cell receptors, and thereby activating the NK cells, and which have been rendered suitable for use in humans, they will generally be assessed for their ability to interact with target cells. In a preferred embodiment, the target cells express an Fc receptor, particularly an Fc gamma receptor such as FCGR3A (also called CD16, FCGR3, Immunoglobulin G Fc Receptor III; IGFR3, Receptor for Fc Fragment of IgG, Low Affinity IIIa,; see, e.g. OMIM 146740), FCGR2A (also called CD32, CDw32, Receptor for Fc Fragment of IgG, Low Affinity IIa, FCG2, Immunoglobulin G Fc Receptor II; see, e.g. OMIM 146790); FCGR2B (also called CD32, Receptor for Fc Fragment of IgG, Low Affinity IIb; FCGR2B, FC-Gamma-RIIB; see, e.g. OMIM 604590), FCG1RA (also called CD64; Receptor for Fc Fragment of IgG, High affinity Ia; IGFR1; see, e.g., OMIM 146760); FCGR1 fragment of IgG, High affinity Ic, Immunoglobulin G Fc receptor IC, IGFRC; see, e.g., OMIM 601503); or FCGR1B (also called CD64, Receptor for Fc Fragment of IgG, High affinity Ib; Immunoglobulin G Fc Receptor IB; IGFRB; see, e.g., OMIM 601502).

The ability of the antibodies to interact with target cells can be assessed using any of a large number of well known methods. In general, the assays described above for detecting antibody binding to activating NK cell receptors such as NKp30, NKp44, and NKp46, including competition-based assays, ELISAs, radioimmunoassays, Western blotting, BIACORE-based assays, and flow cytometry assays, can be equally applied to detect the interaction of humanized, chimeric, or other human-suitable activating NK cell antibodies with target cells. In one embodiment, the target cells used to assess binding are infected or malignant cells selected from the group consisting of B cells, macrophages, mast cells, myeloid cells, monocytes, neutrophils, basophils, eosinophils, langerrhans cells, platelets and endothelial cells of the placenta. In one, preferred embodiment, the target cells are human cells.

In another embodiment, the target cells are cell lines expressing an Fc receptor, such as an Fc gamma receptor such as FCGR3A, FCGR2A, FCGR2B, FCG1RA, FCGR1, or FCGR1B. Again, any suitable assay for measuring the interaction between the antibody—or Fc fragment thereof—and the isolated Fc receptors, or to receptors present within intact cells, lysed cells or membranes or lipid formulations can be performed. Preferably, human cells and/or human Fc receptors will be used in the assays. In one embodiment of any of the assays for detecting binding to target cells, an isolated Fc fragment of the antibody will be used.

In certain embodiments, the antibodies will be further modified to enhance their interaction with Fc receptors, e.g. by altering certain residues within the Fc region that are known to enhance the interaction with Fc receptors, or, using well known methods, to experimentally identify amino acid alterations within the Fc region (or elsewhere within the Ig) that can enhance the binding to Fc receptors. See, e.g., Shields et al. (J. Biol. Chem. 276:6591-6604).

In the present assays, the ability of the humanized or human-suitable antibody, or Fc containing fragment thereof, to bind to the target cell or human Fc receptor will be compared with the ability of a control protein, e.g. a non-human antibody or a non-Ig peptide or protein to bind to the same target. Antibodies or fragments that bind to the target cells or Fc receptor using any suitable assay with 25%, 50%, 100%, 200%, 1000%, or higher increased affinity relative to the control protein, are said to “specifically bind to” or “specifically interact with” the target, and are preferred for use in the therapeutic methods described below.

In one preferred embodiment, therefore, the present invention provides a method for producing an antibody suitable for use in the treatment of a disorder such as a malignancy, including hematological malignancies, or an infection, including viral-infection, the method comprising the following steps: a) providing a monoclonal antibody that binds specifically to and activates a human activating NK cell receptor; b) humanizing the antibody or otherwise rendering it suitable for use in humans; and c) assessing the ability of the humanized or human-suitable antibody to specifically interact with human Fc receptor expressing target cells or with an isolated human Fc receptor, wherein a determination that the humanized or human-suitable antibody binds specifically to the human Fc receptor expressing target cell and/or to an isolated human Fc receptor indicates that the antibody is suitable for use in the treatment of the disorder.

It will be appreciated that equivalent methods can be used to produce antibodies suitable for treating animals, or for testing in an animal model. In that case, the antibodies will be ensured to be capable of specifically recognizing and activating NK cell receptors from the relevant animal, and to be recognized by Fc receptors from the same animal. Similarly, the antibody will be modified to be suitable for administration into the animal.

In certain embodiments, the antibodies will be evaluated in vivo, in suitable animal models. For example, mouse models containing human NK cells are administered human-suitable antibodies, and the activity of the human NK cells in the mouse is addressed (in terms of cytokine production, cytotoxic activity, etc.) In a preferred embodiment, the animal model contains both human NK cells and human tumor or infected cells, preferably Fc gamma receptor positive tumor or infected cells, and the ability of the antibody to stimulate the activation of the NK cells, interact with the target cells, and/or lyse, kill, reduce the number of, inhibit the growth of, or otherwise inhibit the target cells is determined. See, e.g., Example 7.

Administration of Antibodies and Identification of Candidates for Treatment Methods

The antibodies produced using the present methods are particularly effective at treating certain disorders, such as cancers, solid and non solid tumors, and hematological malignancies, as well as for infections such as viral infections. In a preferred embodiment, the present antibodies are useful for treating patients with tumors or other malignancies, viral infections, or any other presence of undesired cells, wherein the malignant, infected, or undesired cells express one or more Fc receptor such as Fc gamma receptors. In one embodiment, the cells are MHC class I-defective.

The present invention also provides methods for identifying patients suitable for treatment using the present antibodies, or for identifying individuals suitable for inclusion in a clinical trial designed to assess the therapeutic efficacy of an antibody of the present invention. In particular, individuals having malignancies such as hematological malignancies, or virally infected cells, wherein the malignancy or virally-infected cells include Fc receptor-expressing cells, such as Fc gamma receptors, are particularly well suited for such treatments or for inclusion in such a clinical trial. Accordingly, the present invention provides a method for treating a patient with a malignancy or infection, or for identifying patients with a malignancy or infection that are suitable for inclusion in a clinical trial, comprising determining whether the malignancy or infection includes Fc receptor expressing cells, wherein a determination that the malignancy or infection includes Fc receptor expressing cells indicates that the individual is suited for treatment or for inclusion in the clinical trial, where the treatment or clinical trial involves the administration of the present antibodies. In a preferred embodiment, the Fc receptor is an Fc gamma receptor. In another preferred embodiment, the malignancy is a hematological malignancy such as follicular lymphoma, B cell lymphoma, macrophage tumors, acute monocytic leukemia, chronic myelomonocytic leukemia, chronic lymphocytic leukemia, mast cell malignancy, myeloid metaplasia, or chronic myelocytic leukemia, or, more generally, the malignancy or infection involves any of the following cell types: B cells, macrophages, mast cells, myeloid cells, monocytes, neutrophils, basophils, eosinophils, langerrhans cells, platelets and endothelial cells of the placenta.

In such methods, the presence of Fc receptor expressing cells can be detected in any of a large number of ways well known to those of skill in the art. For example, a biopsy can be performed to isolate tumor cells or infected cells, and the presence or absence of Fc receptors on the surface of the cells can be detected using antibodies that specifically recognize one or more Fc receptors. Such antibodies are readily available, and methods for detecting specific receptors on cells are well known in the art. Any detection of any Fc receptor indicates that the patient is a candidate for treatment using the present antibodies, or suitable for inclusion in a clinical trial to assess the efficacy of any particular antibody produced using the present methods.

The invention also provides compositions, e.g., pharmaceutical compositions, that comprise any of the present antibodies, including fragments and derivatives thereof, in any suitable vehicle in an amount effective to detectably enhance NK cell cytotoxicity in a patient or in a biological sample comprising NK cells, and enhance the ability of the cytotoxic NK cells to target tumor, virally-infected, or Fc receptor expressing cells. The composition generally further comprises a pharmaceutically acceptable carrier. Isolated antibodies of the present invention are also provided, as are kits comprising the present antibodies, e.g., for use in treatment methods.

Pharmaceutically acceptable carriers that may be used in these compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of this invention may be employed in a method of enhancing the activity of NK cells in a patient or a biological sample. For use in conjunction with a patient, the composition must be formulated for administration to the patient. It will be appreciated that the present methods of administering antibodies and compositions to patients can also be used to treat animals, or to test the efficacy of any of the herein-described methods or compositions in animal models for human diseases.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

In another embodiment, the antibodies of the present invention can be used as part of a grafting method for treating malignancies and infections. For example, a patient is provided with purified NK cells that have been activated according to the methods of the invention. This notably applies to MHC-matched or -mismatched hematopoietic grafts (bone marrow/peripheral stem cells). These activated NK cells, e.g. allogeneic NK cells, can, e.g., be intravenously infused into the patient to be grafted, and ideally no later than about 2 days after the graft. In one embodiment, autologous NK cells from a patient suffering from solid or liquid tumors, or from a viral or other micro-organism infection, are purified and activated according to the methods and/or compositions of the invention, and then re-injected intravenously into the patient. The precise amount and frequency of infusion into patients will depend upon the particular pathology and will most likely be best determined on a case-by-case basis by a qualified medical practitioner.

Sterile injectable forms of the compositions of this invention may be aqueous or an oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

In one embodiment, the antibodies of this invention may be incorporated into liposomes (“immunoliposomes”), alone or together with another substance for targeted delivery to a patient or an animal. Such other substances can include nucleic acids for the delivery of genes for gene therapy or for the delivery of antisense RNA, RNAi or siRNA for suppressing a gene in an NK cell, or toxins or drugs for the activation of NK cells through other means, or any other agent described herein that may be useful for activation of NK cells or targeting of tumor or infected cells.

The compositions of this invention may also be administered topically, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract can be effected in a rectal suppository formulation (see above) or in a suitable enema formulation. Topically-transdermal patches may also be used.

For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions may be formulated in an ointment such as petrolatum.

The compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Several monoclonal antibodies have been shown to be efficient in clinical situations, such as Rituxan (Rituximab), Herceptin (Trastuzumab) or Xolair (Omalizumab), and similar administration regimens (i.e., formulations and/or doses and/or administration protocols) may be used with the antibodies of this invention. Schedules and dosages for administration can be determined in accordance with known methods for these products, for example using the manufacturers' instructions. For example, a monoclonal antibody can be supplied at a concentration of 10 mg/mL in either 100 mg (10 mL) or 500 mg (50 mL) single-use vials. The product is formulated for IV administration in 9.0 mg/ML sodium chloride, 7.35 mg/ML sodium citrate dihydrate, 0.7 mg/mL polysorbate 80, and Sterile Water for Injection. The pH is adjusted to 6.5. An exemplary suitable dosage range for cross-reacting KIR antibody of the invention may between about 10 mg/m2 and 500 mg/m2. However, it will be appreciated that these schedules are exemplary and that optimal schedule and regimen can be adapted taking into account the affinity of the antibody and the tolerability of the NK cell activating antibodies that must be determined in clinical trials. Quantities and schedule of injection of antibodies to activating NK cell receptors that saturate NK cells for 24 hours, 48 hours 72 hours or a week or a month will be determined considering the affinity of the antibody and the its pharmacokinetic parameters.

According to another embodiment, the antibody compositions of this invention may further comprise another therapeutic agent, including agents normally utilized for the particular therapeutic purpose for which the antibody is being administered. The additional therapeutic agent will normally be present in the composition in amounts typically used for that agent in a monotherapy for the particular disease or condition being treated. Such therapeutic agents include, but are not limited to, therapeutic agents used in the treatment of cancers, therapeutic agents used to treat infectious disease, therapeutic agents used in other immunotherapies, cytokines (such as IL-2 or IL-15), other antibodies and fragments of other antibodies.

For example, a number of therapeutic agents are available for the treatment of cancers. The antibody compositions and methods of the present invention may be combined with any other methods generally employed in the treatment of the particular disease, particularly a tumor, cancer, malignancy, or other disease or disorder that the patient exhibits. So long as a particular therapeutic approach is not known to be detrimental to the patient's condition in itself, and does not significantly counteract the activating NK receptor antibody-based treatment, its combination with the present invention is contemplated.

In connection with solid tumor treatment, the present invention may be used in combination with classical approaches, such as surgery, radiotherapy, chemotherapy, and the like. The invention therefore provides combined therapies in which humanized or human-suitable antibodies against activating NK receptors are used simultaneously with, before, or after surgery or radiation treatment; or are administered to patients with, before, or after conventional chemotherapeutic, radiotherapeutic or anti-angiogenic agents, or targeted immunotoxins or coaguligands.

When one or more agents are used in combination with the present antibody-based therapy, there is no requirement for the combined results to be additive of the effects observed when each treatment is conducted separately. Although at least additive effects are generally desirable, any increased anti-cancer effect above one of the single therapies would be of benefit. Also, there is no particular requirement for the combined treatment to exhibit synergistic effects, although this is certainly possible and advantageous.

To practice combined anti-cancer therapy, one would simply administer to a patient an activating NK receptor antibody composition in combination with another anti-cancer agent in a manner effective to result in their combined anti-cancer actions within the patient. The agent would therefore be provided in amounts effective and for periods of time effective to result in their combined presence within the tumor vasculature and their combined actions in the tumor environment. To achieve this goal, the activating NK receptor antibody-based therapeutic and anti-cancer agents may be administered to the patient simultaneously, either in a single composition, or as two distinct compositions using different administration routes.

Alternatively, the activating NK receptor antibody-based treatment may precede, or follow, the anti-cancer agent treatment by, e.g., intervals ranging from minutes to weeks and months. One would ensure that the anti-cancer agent and activating NK cell receptor-based agent exert an advantageously combined effect on the cancer.

Most anti-cancer agents would be given prior to activating NK receptor-based anti-angiogenic therapy. However, where activating NK receptor-based immunoconjugates are used, various anti-cancer agents may be simultaneously or subsequently administered.

In some situations, it may even be desirable to extend the time period for treatment significantly, where several days (e.g., 2, 3, 4, 5, 6 or 7), several weeks (e.g., 1, 2, 3, 4, 5, 6, 7 or 8) or even several months (e.g., 1, 2, 3, 4, 5, 6, 7 or 8) lapse between the respective administrations. This would be advantageous in circumstances where one treatment was intended to substantially destroy the tumor, such as surgery or chemotherapy, and another treatment such as with an activating NK cell receptor antibody composition was intended to prevent micrometastasis or tumor re-growth.

It also is envisioned that more than one administration of either the activating NK cell receptor antibody-based composition or the anti-cancer agent will be utilized. The agents may be administered interchangeably, on alternate days or weeks; or a cycle of activating NK cell receptor antibody-based treatment may be given, followed by a cycle of anti-cancer agent therapy. In any event, to achieve tumor regression using a combined therapy, all that is required is to deliver both agents in a combined amount effective to exert an anti-tumor effect, irrespective of the times for administration.

In terms of surgery, any surgical intervention may be practiced in combination with the present invention. In connection with radiotherapy, any mechanism for inducing DNA damage locally within cancer cells is contemplated, such as gamma-irradiation, X-rays, UV-irradiation, microwaves and even electronic emissions and the like. The directed delivery of radioisotopes to cancer cells is also contemplated, and this may be used in connection with a targeting antibody or other targeting means.

In other aspects, immunomodulatory compounds or regimens may be practiced in combination with the present invention. Preferred examples include treatment with cytokines. Various cytokines may be employed in such combined approaches. Examples of cytokines include IL-1alpha IL-1beta, IL-2, IL-3, IL-4, IL-5, IL-6, IL-7, IL-8, IL-9, IL-10, IL-11, IL-12, IL-13, IL-15, IL-21, TGF-beta, GM-CSF, M-CSF, G-CSF, TNF-alpha, TNF-beta, LAF, TCGF, BCGF, TRF, BAF, BDG, MP, LIF, OSM, TMF, PDGF, IFN-alpha, IFN-beta, IFN-gamma. Particularly preferred are cytokines that stimulate NK cell cytotoxic activity, such as IL-2, IL-12, or IL-15. Cytokines are administered according to standard regimens, consistent with clinical indications such as the condition of the patient and relative toxicity of the cytokine.

Other preferred examples of compounds or regimens that may be practiced in combination with those of the present invention include compositions modulating the activity of NK cells. For example, in certain, preferred embodiments, the antibodies of the present invention will be administered in conjunction with compounds capable of interfering with NK cell inhibitory receptors, such as antibodies or small molecules that can inhibit the activity of CD94/NKG2A receptors or a proinflammatory HLA-E binding peptide as described in PCT Publication no. WO 03/011895, the disclosure of which is incorporated herein by reference, or any KIR receptors that inhibit NK cell activation, as described in, e.g., (Yawata et al. (2002) Crit Rev immunol. 22(5-6):463-82; Middleton et al. (2002) Transpl Immunol. 10(2-3):147-64; Vilches et al. (2002) Annu Rev Immunol. 20:217-51; or Long et al. 2001 Immunol Rev. 181:223-33), the disclosures of each of which are herein incorporated by reference in their entireties.

In certain embodiments, the activating NK cell receptor antibody therapeutic compositions of the present invention may be administered in combination with a chemotherapeutic or hormonal therapy agent. A variety of hormonal therapy and chemotherapeutic agents may be used in the combined treatment methods disclosed herein. Chemotherapeutic agents contemplated as exemplary include alkylating agents, antimetabolites, cytotoxic antibiotics, vinca alkaloids, for example adriamycin, dactinomycin, mitomycin, carminomycin, daunomycin, doxorubicin, tanioxifen, taxol, taxotere, vincristine, vinblastine, vinorelbine, etoposide (VP-16), 5-fluorouracil (5FU), cytosine arabinoside, cyclophosphamide, thiotepa, methotrexate, camptothecin, actinomycin-D, mitomycin C, cisplatin (CDDP), aminopterin, combretastatin(s) and derivatives and prodrugs thereof. Hormonal agents include for example LHRH agonists such as leuprorelin, goserelin, triptorelin, and buserelin; anti-estrogens such as tamoxifen and toremifene; anti-androgens such as flutamide, nilutamide, cyproterone and bicalutamide; aromatase inhibitors such as anastrozole, exemestane, letrozole and fadrozole; and progestagens such as medroxy, chlormadinone and megestrol.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating may be used. Cisplatin has been widely used to treat cancer, with efficacious doses used in clinical applications of 20 mg/m.sup.2 for 5 days every three weeks for a total of three courses. Cisplatin is not absorbed orally and must therefore be delivered via injection intravenously, subcutaneously, intratumorally or intraperitoneally.

Further useful agents include compounds that interfere with DNA replication, mitosis and chromosomal segregation, and agents that disrupt the synthesis and fidelity of polynucleotide precursors may also be used. A number of exemplary chemotherapeutic agents for combined therapy are listed in Table C of U.S. Pat. No. 6,524,583, the disclosure of which agents and indications are specifically incorporated herein by reference. Each of the agents listed are exemplary and not limiting. The skilled artisan is directed to “Remington's Pharmaceutical Sciences” 15th Edition, chapter 33, in particular pages 624-652. Variation in dosage will likely occur depending on the condition being treated. The physician administering treatment will be able to determine the appropriate dose for the individual subject.

The present antibodies may be used in combination with any one or more other anti-angiogenic therapies. Examples of such agents include neutralizing antibodies antisense strategies, RNA aptamers and ribozymes against VEGF or VEGF receptors (U.S. Pat. No. 6,524,583, the disclosure of which is incorporated herein by reference). Variants of VEGF with antagonistic properties may also be employed, as described in WO 98/16551, specifically incorporated herein by reference. Further exemplary anti-angiogenic agents that are useful in connection with combined therapy are listed in Table D of U.S. Pat. No. 6,524,583, the disclosure of which agents and indications are specifically incorporated herein by reference.

Activating NK cell antibodies may also be advantageously combined with methods to induce apoptosis. For example, a number of oncogenes have been identified that inhibit apoptosis, or programmed cell death. Exemplary oncogenes in this category include, but are not limited to, bcr-ab1, bcl-2 (distinct from bcl-1, cyclin D1; GenBank accession numbers M14745, X06487; U.S. Pat. Nos. 5,650,491; and 5,539,094; each incorporated herein by reference) and family members including Bcl-x1, Mcl-1, Bak, A1, A20. Overexpression of bcl-2 was first discovered in T cell lymphomas. bcl-2 functions as an oncogene by binding and inactivating Bax, a protein in the apoptotic pathway. Inhibition of bcl-2 function prevents inactivation of Bax, and allows the apoptotic pathway to proceed. Ihibition of this class of oncogenes, e.g., using antisense nucleotide sequences or small molecule chemical compounds, is contemplated for use in the present invention to give enhancement of apoptosis (U.S. Pat. Nos. 5,650,491; 5,539,094; and 5,583,034; each incorporated herein by reference).

The present antibodies may also be used in combination with moieties in which the targeting portion thereof, e.g., antibody or ligand, is directed to a relatively specific marker of a target cell, for example a target tumor cell. Generally speaking, antibodies or ligands for use in these additional aspects of the invention will preferably recognize accessible tumor antigens that are preferentially, or specifically, expressed in the tumor site. The targeting agents will generally bind to a surface-expressed, surface-accessible or surface-localized component of a tumor cell. The antibodies or ligands will also preferably exhibit properties of high affinity; and the antibodies, ligands or conjugates thereof, will not exert significant in vivo side effects against life-sustaining normal tissues, such as one or more tissues selected from heart, kidney, brain, liver, bone marrow, colon, breast, prostate, thyroid, gall bladder, lung, adrenals, muscle, nerve fibers, pancreas, skin, or other life-sustaining organ or tissue in the human body. The term “significant side effects”, as used herein, refers to an antibody, ligand or antibody conjugate, that, when administered in vivo, will produce only negligible or clinically manageable side effects, such as those normally encountered during chemotherapy.

In the treatment of tumors, any therapy involving the activating NK cell antibodies may be used in combination with adjunct compounds. Adjunct compounds may include by way of example anti-emetics such as serotonin antagonists and therapies such as phenothiazines, substituted benzamides, antihistamines, butyrophenones, corticosteroids, benzodiazepines and cannabinoids; bisphosphonates such as zoledronic acid and pamidronic acid; and hematopoietic growth factors such as erythropoietin and G-CSF, for example filgrastim, lenograstim and darbepoietin.

In another embodiment, two or more antibodies of this invention having different specificities may be combined in a single composition so as to activate as many NK cells or types of NK cells as possible, and/or to target as many NK cell target cell types as possible. Compositions comprising combinations of the present antibodies, or fragments or derivatives thereof, will allow even wider utility because there likely exists a small percentage of the human population that may lack one of the activating receptors, or a specific epitope within one of the receptors, that is recognized by a single activating antibody.

The invention also provides a method of activating NK cell activity in a patient in need thereof, comprising the step of administering a composition according to this invention to said patient. The method is more specifically directed at increasing NK cell activity in patients having a disease in which increased NK cell activity is beneficial, which involves, affects or is caused by cells susceptible to lysis by NK cells, or which is caused or characterized by insufficient NK cell activity, such as a cancer, an infectious disease or an immune disorder. More specifically, the methods of the present invention are utilized for the treatment of a variety of cancers and other proliferative diseases including, but not limited to, carcinoma, including that of the bladder, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma.

Preferred disorders include hematological malignancies that comprise cells that express one or more Fc receptors, preferably Fc gamma receptors, including, but not limited to, hematopoietic tumors of lymphoid lineage, for example T-cell and B-cell tumors, including but not limited to T-cell disorders such as T-prolymphocytic leukemia (T-PLL), including of the small cell and cerebriform cell type; Large granular lymphocyte leukemia (LGL) preferably of the T-cell Type; Sezary syndrome (SS); Adult T-cell leukemia lymphoma (ATLL); a/d T-NHL hepatosplenic lymphoma; Peripheral/post-thymic T cell lymphoma (pleomorphic and immunoblastic subtypes); Angio immunoblastic T-cell lymphoma; Angiocentric (nasal) T-cell lymphoma; Anaplastic (Ki 1+) large cell lymphoma; Intestinal T-cell lymphoma; and T-lymphoblastic; Lymphoma/leukemia (T-Lbly/T-ALL). Most preferred are follicular lymphoma, B cell lymphoma, macrophage tumors, acute monocytic leukemia, chronic myelomonocytic leukemia, chronic lymphocytic leukemia, mast cell malignancy, myeloid metaplasia, and chronic myelocytic leukemia, or any malignancy or proliferative disorder involving B cells, macrophages, mast cells, myeloid cells, monocytes, neutrophils, basophils, eosinophils, langerrhans cells, platelets and endothelial cells of the placenta.

Other proliferative disorders can also be treated according to the invention, including for example hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. The activating NK cell antibody-based treatment can be used to treat or prevent infectious diseases, including preferably any infections caused by infection by viruses, bacteria, protozoa, molds or fungi. Such viral infectious organisms include, but are not limited to, hepatitis type A, hepatitis type B, hepatitis type C, influenza, varicella, adenovirus, herpes simplex type I (HSV-1), herpes simplex type 2 (HSV-2), rinderpest, rhinovirus, echovirus, rotavirus, respiratory syncytial virus, papilloma virus, papilloma virus, cytomegalovirus, echinovirus, arbovirus, huntavirus, coxsackie virus, mumps virus, measles virus, rubella virus, polio virus and human immunodeficiency virus type I or type 2 (HIV-1, HIV-2). Bacteria constitute another preferred class of infectious organisms including but are not limited to the following: Staphylococcus; Streptococcus, including lo S. pyogenes; Enterococcl; Bacillus, including Bacillus anthracis, and Lactobacillus; Listeria; Corynebacterium diphtheriae; Gardnerella including G. vaginalis; Nocardia; Streptomyces; Thermoactinomyces vulgaris; Treponema; Camplyobacter, Pseudomonas including Raeruginosa; Legionella; Neisseria including N. gonorrhoeae and N. meningitides; Flavobacterium including F. meningosepticum and F. odoratun; Brucella; Bordetella including B. pertussis and B. bronchiseptica; Escherichia including E. coli, Klebsiella; Enterobacter, Serratia including S. marcescens and S. liquefaciens; Edwardsiella; Proteus including P. mirabilis and P. vulgaris; Streptobacillus; Rickettsiaceae including R. fickettsfi, Chlamydia including C. psittaci and C. trachomatis; Mycobacterium including M. tuberculosis, M. intracellulare, M. folluiturn, M. laprae, M. avium, M. bovis, M. africanum, M. kansasii, M. intracellulare, and M. lepraemurium; and Nocardia. Protozoa may include but are not limited to, leishmania, kokzidioa, and trypanosoma. Parasites include but are not limited to, chlamydia and rickettsia. A complete list of infectious diseases can be found on the website of the National Center for Infectious Disease (NCID) at the Center for Disease Control (CDC) (worldwide web site www.cdc.gov/ncidod/diseases/), which list is incorporated herein by reference. Also treatable are infections of cell types including B cells, macrophages, mast cells, myeloid cells, monocytes, neutrophils, basophils, eosinophils, langerrhans cells, platelets and endothelial cells of the placenta. All of said diseases are candidates for treatment using the antibodies of the invention.

### EXAMPLES

Further aspects and advantages of this invention are disclosed in the following experimental section, which should be regarded as illustrative and not limiting the scope of this application.

### Example 1

### Generation of mAbs Specific to Activating NK Cell Receptors

Novel monoclonal antibodies are generated by immunizing 5 week old Balb C mice with activated polyclonal or monoclonal NK cell lines, e.g., as described in Moretta et al. (1990) J Exp Med. 172(6):1589-98. After different cell fusions, the mAbs are first selected for their ability to specifically recognize one or more activating NK cell receptors, such as NKp30, NKp44, and NKp46. Positive monoclonal antibodies are further screened for their ability to reconstitute lysis by EB6 positive or GL183 positive NK clones of Cw4 or Cw3 positive targets respectively, or in redirected killing assays against P815 target cells. Appropriate mAbs include those which induce a statistically significant (p<0.05) increase in NK cell activation as assessed by (i) natural cytotoxicity towards MHC class I negative targets, tumor cells, virally-infected cells, allogeneic cells, (ii) cytotoxicity towards antibody-coated target cells, (iii) increases in intracytoplasmic Ca2+ concentration, (iv) induction of tyrosine phosphorylation of intracytoplasmic adaptor/effector molecules such as ZAP70, Syk, LAT, SLP76, Shc, Grb2, phospholipase C-gamma enzymes, phosphatidyl-inositol 3-kinases, (vi) phosphorylation of receptor-associated transducing chains KARAPIDAP12 or CD3zeta or FcRgamma, (vi) cytokine secretion such as interferon gamma, tumor necrosis factors, IL5, IL10, chemokines (such as MIP-1alpha), TGF-beta, (vii) up- or down-regulation of NK cell surface molecules, such as CD69 and PEN5 respectively. Preferred mAbs induce e.g. an induce an increase of at least about 5 in target cell lysis with an effector:target (E:T) ratio of 1:1 when compared to the basic target cell lysis performed by the effector NK cells in the absence of said mAbs.

### Example 2

### Purification of Peripheral Blood Lymphocytes (PBL) and Generation of Polyclonal or Clonal NK Cell Populations

Peripheral blood lymphocytes (PBL) are derived from healthy donors by Ficoll-Hipaque gradients and depletion of plastic-adherent cells. In order to obtain enriched NK cells PBL are incubated with anti-CD3 (JT3A), anti-CD4 (HP2.6) and anti-HLA-DR (D1.12) mAbs (30 min at 4 degrees C.) followed by goat anti-mouse coated Dynabeads (Dynal, Oslo, Norway) (30 min at 4 degrees C.) and immunomagnetic depletion (Pende et al. (1998) Eur. J. Immunol. 28:2384-2394; Sivori et al. (1997) J. Exp. Med. 186: 1129-1136; Vitale et al. (1998) J. Exp. Med. 187:2065-2072). CD3⁻4⁻DR⁻ cells are used in cytolytic assays or cultured on irradiated feeder cells in the presence of 100 U/ml rlL-2 (Proleukin, Chiron Corp., Emeryville, USA) and 1.5 ng/ml PHA (Gibco Ltd, Paisley, Scotland) in order to obtain polyclonal NK cell populations or, after limiting dilution), NK cell clones (Moretta (1985) Eur. J. Immunol. 151:148-155).

### Example 3

### Flow Cytofluorimetric Analysis

Cells are stained with the appropriate mAb followed by PE- or FITC-conjugated isotype-specific goat anti-mouse second reagent (Southern Biotechnology Associated, Birmingham, Ala.). Samples are analyzed by one- or two-color cytofluorimetric analysis (FACScan Becton Dickinson & Co, Mountain View, Calif.) as previously described (e.g. Moretta et al. (1990) J. Exp. Med. 171:695-714).

### Example 4

### Cell Lines and Cytolytic Assays

The Fc gamma receptor negative targets used are the following: MEL15 (MEL15392, human melanoma) (Pende et al. (1998) Eur J. Immunol. 28:2384-2394); M14 (human melanoma; Pessino et al (1998) J. Exp. Med. 188:953-960); SMMC (human hepatocarcinoma; Sivori et al. (1999) Eur J. Immunol. 29:1656-1666); A549 (human lung adenocarcinoma; ATCC number CCL-185.1); FO-1 and 1174 mel (human melanomas); AUMA (human melanoma). The Fc gamma receptor positive EBV transformed cell lines such as 721.221 are used. EBV and normal target cells, are obtained by culturing PBL with 1.5 ng/ml PHA (Gibco).

Cells are tested for cytolytic activity in a 4-h ⁵¹Cr-release assay as previously described), either in the absence or in the presence of various mAbs (Moretta et al. (1990) J. Exp. Med. 172:1589-1598; Sivori et al. (1999) Eur. J. Immunol. 29:1656-1666). The concentrations of the various mAbs are 10 μg/ml for the masking experiments and 0.5 μg/ml for the redirected killing experiments. Appropriate mAbs include those which significantly increase the cytolytic activity observed in their absence. Examples of such an appropriate significant increase comprise an increase of at least about 5 times of the cytolytic activity observed with an effector:target ratio of 1:1 in the presence of said mAbs when compared to the cytolytic activity observed in the absence of these mAbs.

### Example 5

### Determination of Intracellular Free Calcium [Ca⁺⁺]i Increase

Determination of [Ca⁺⁺]i is performed as previously described (Poggi et al. (1993) Eur. J. Immunol. 23:2445-2463). Fura-2-labeled NK cells are incubated for 30″ at 4 degrees C. with saturating amounts of the relevant antibody, e.g., anti-NKp46 antibody (BAB281), anti-NKp30 mAb (AZ20), etc., or medium alone. Cross-inking of the receptor can be obtained by adding into the cuvette 20 μg/ml of affinity purified Goat Anti-Mouse antiserum (GAM) (ICN Biomedicals, Aurora, Ohio).

### Example 6

### Biacore Analysis of Antibody-Substrate Interactions

Production and Purification of Recombinant Proteins

The recombinant proteins are produced in E. coli. cDNA encoding the entire extracellular domain of an NK cell activating receptor, which had been amplified by PCR using standard methods. The nucleic acid sequences are cloned into the pML1 expression vector in frame with a sequence encoding a biotinylation signal (Saulquin et al, 2003).

Protein expression is performed in the BL21(DE3) bacterial strain (Invitrogen). Transfected bacteria were grown to OD₆₀₀=0.6 at 37° C. in medium supplemented with ampicillin (100 μg/ml) and expression was induced with 1 mM IPTG.

Proteins are recovered from inclusion bodies under denaturing conditions (8 M urea). Refolding of the recombinant proteins is performed in 20 mM Tris, pH 7.8, NaCl 150 mM buffer containing L-argliine (400 mM, Sigma) and P-mercaptoethanol (1 mM), at room temperature, by decreasing the urea concentration in a six step dialysis (4, 3, 2, 1 0.5 and 0 M urea, respectively). Reduced and oxidized glutathione (5 mM and 0.5 mM respectively, Sigma) are added during the 0.5 and 0 M urea dialysis steps. Finally, the proteins are dialyzed extensively against 10 mM Tris, pH 7.5, NaCl 150 mM buffer. Soluble, refolded proteins were concentrated and then purified on a Superdex 200 size-exclusion column (Pharmacia; AKTA system).

Surface plasmon resonance measurements are performed on a Biacore apparatus (Biacore). In all Biacore experiments HBS buffer supplemented with 0.05% surfactant P20 served as running buffer.

Protein Immobilization

Recombinant substrate proteins produced as described above are immobilized covalently to carboxyl groups in the dextran layer on a Sensor Chip CM5 (Biacore). The sensor chip surface is activated with EDC/NHS (N-ethyl-N′-(3-dimethylaminopropyl)carbodiimidehydrochloride and N-hydroxysuccinimide, Biacore). Proteins, in coupling buffer (10 mM acetate, pH 4.5) were injected. Deactivation of the remaining activated groups was performed using 100 mM ethanolamine pH 8 (Biacore).

Affinity Measurements

For kinetic measurements, various concentrations of the soluble antibody (1×10⁻⁷ to 4×10⁻¹⁰ M) are applied onto the immobilized substrate ample. Measurements are performed at a 20 μl/min continuous flow rate. For each cycle, the surface of the sensor chip is regenerated by 5 μl injection of 10 mM NaOH pH 11. The BIAlogue Kinetics Evaluation program (BIAevaluation 3.1, Biacore) is used for data analysis. The soluble analyte (40 μl at various concentrations) is injected at a flow rate of 20 μl/min in HBS buffer, on dextran layers containing, e.g. 500 reflectance units (RU), and 1000 RU, of substrate.

### Example 7

### Testing Human-Suitable Antibodies In Vivo in a Mouse Model

Preliminary studies to set up the Nod-SCID/tumor and to stimulate human MNC with anti-NCR antibody in the Nod-SCID are carried out as follows:

EBV transformed cell lines such as line 721.221 can be used. 8-12 week-old Nod-SCID mice are used as recipients of the cells lines. Other cell lines may be selected and used accordingly.

Each mouse receives, at the left flank's upper part, 2-5×10⁶ cells by subcutaneous injection at day D0. The appearance and growth of the tumors evolution are checked weekly.

Tumor appearance and development depends on the cell line and on the number of engrafted cells. For example, after the engraftment of 2×10⁶ cells, 3 to 4 weeks may be needed for the 786-O and Kaci-1, while 2 weeks may be needed for the G401 and 402. When the volume of the solid tumor reaches more than >30 mm3 (calculated with the formula A2×B/2 where A and B represent respectively the length and breadth of the tumor), the mice, randomized, receive intraperitoneal (IP) PBMC and receive anti-NCR antibody treatment.

At day 0, mice are injected IP 50×10⁶ PBMC (collected from healthy donor, etablissement frangais du sang), then stimulated by (IP) injection of about 200 μg of anti-NCR antibody. IL2 (1000 IU) is administered conjointly with the anti-NCR antibody, and thereafter daily (500 IU) for 5 days. If desired, the development of human NK cells in the peritoneal cavity of Nod-SCID/human (hu) anti-NCR antibody-treated mice is then assessed using any suitable method, for example flow cytometry-based methods.

Several groups are constituted:
Negative control group: Mice in the group receive only tumoral cell line
PBMC group: Mice are injected IP with 50×10⁶ PBMC
Study group: Mice after receiving the 50×10⁶ PBMC are treated with anti-NCR antibody and IL2.

For each group, the tumor size and volume is checked weekly.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-24. (canceled)

25. A method for producing an antibody suitable for use in treating hematological malignancies, said method comprising a) providing a human-suitable antibody that specifically binds to a human activating NK cell receptor, and b) assessing the ability of said antibody to specifically bind to a human Fc receptor, wherein a determination that said antibody can specifically bind to said receptor indicates that said antibody is suitable for use in treating said malignancies.

26. The method of claim 25, wherein said human Fc receptor is an Fc gamma receptor, and wherein said antibody is an IgG.

27. The method of claim 26, wherein said Fc gamma receptor is selected from the group consisting of FCGR1A, FCGR1B, FCGR2A, FCGR2B, and FCGR3.

28. The method of claim 25, wherein said Fc receptor is present on the surface of a cell or present within a cell membrane.

29. The method of claim 25, wherein said hematological malignancy is selected from the group consisting of follicular lymphoma, B cell lymphoma, macrophage tumors, acute monocytic leukemia, chronic myelomonocytic leukemia, chronic lymphocytic leukemia, mast cell malignancy, myeloid metaplasia, and chronic myelocytic leukemia.

30. The method of claim 25, wherein human-suitable antibody is derived from a mouse monoclonal antibody.

31. The method of claim 25, wherein said activating NK cell receptor is NKp30, NKp44, or NKp46.

32. The method of claim 31, wherein said activating NK cell receptor is NKp30, and wherein said human-suitable antibody is derived from a mouse monoclonal antibody selected from the group consisting of AZ20, A76, and Z25.

33. The method of claim 31, wherein said NK cell activating receptor is NKp44, and wherein said human-suitable antibody is derived from mouse monoclonal antibody Z231.

34. The method of claim 31, wherein said NK cell activating receptor is NKp46, and wherein said human-suitable antibody is derived from mouse monoclonal antibody BAB281.

35. A pharmaceutical composition comprising an antibody produced by the method of claim 25, and a pharmaceutically acceptable carrier.

36. A method for treating a patient with a hematological malignancy selected from the group consisting of follicular lymphoma, B cell lymphoma, macrophage tumors, acute monocytic leukemia, chronic myelomonocytic leukemia, chronic lymphocytic leukemia, mast cell malignancy, myeloid metaplasia, and chronic myelocytic leukemia, said method comprising administering to said patient a pharmaceutical composition comprising a human-suitable antibody that specifically binds to a human activating NK cell receptor, and a pharmaceutically acceptable carrier.

37. The method of claim 36, wherein said human activating NK cell receptor is selected from the group consisting of NKp30, NKp44, and NKp46.

38. The method of claim 37, wherein said activating NK cell receptor is NKp30, and wherein said human-suitable antibody is derived from a mouse monoclonal antibody selected from the group consisting of AZ20, A76, and Z25.

39. The method of claim 37, wherein said receptor is NKp44, and wherein said human-suitable antibody is derived from the monoclonal antibody Z231.

40. The method of claim 37, wherein said receptor is NKp46, and wherein said human-suitable antibody is derived from the monoclonal antibody BAB281.

41. The method of claim 36, wherein said antibody is an IgG.

42. The method of claim 41, wherein said malignancy comprises cells expressing an Fc gamma receptor.

43. The method of claim 42, wherein said Fc gamma receptor is selected from the group consisting of FCGR1A, FCGR1B, FCGR2A, FCGR2B, and FCGR3.

44. The method of claim 36, further comprising a step in which the ability of said human-suitable antibody to interact with malignant cells taken from said patient is assessed prior to the administration of said composition, and wherein a determination that said antibody specifically binds to said malignant cells indicates that said antibody is suitable for use in said treatment.

45. The method of claim 36, further comprising a step in which the presence or absence of Fc receptors on the surface of malignant cells taken from said patient is assessed, wherein a detection of an Fc receptor on the surface of said malignant cells indicates that said antibody is suitable for use in said treatment.

46. The method of claim 45, wherein said Fc receptor is an Fc gamma receptor.

47. The method of claim 36, wherein said human-suitable antibody contains a human Fc region.

48. The method of claim 36, wherein said human-suitable antibody is a humanized or chimeric antibody.
